(19) United States
(12) Patent Application Publication
(10) Pub. No.: US 2026/0334964 A1
(43) Pub. Date: Oct. 29, 2026

(54) PULL-PUSH CIRCUITS FOR CLOT EXTRACTION SYSTEMS AND CLOT EXTRACTION METHODOLOGIES

(71) Applicant: JUPITER ENDOVASCULAR INC., Menlo Park, CA (US)

(72) Inventors: Garrett J. Gomes, Pleasant Hills, CA (US); Jeff Haydon, San Mateo, CA (US)

(21) Appl. No.: 19/574,042

(22) Filed: Mar. 20, 2026

Related U.S. Application Data
(60) Provisional application No. 63/794,251, filed on Apr. 24, 2025.

(51) Int. Cl. A61B 17/22; A61B 17/00

(52) U.S. Cl. CPC A61B 17/22; A61B 17/00234; A61B 2017/00137; A61B 2017/00398; A61B 2017/00544; A61B 2017/00561; A61B 2017/00778; A61B 2017/22079; A61B 2217/005

(57) ABSTRACT

Circuits, systems, and methods for treatment of clots, such as emboli and/or thrombi from a blood vessel of a patient, are provided. The configurations of a pull-push circuit allow a pump to apply suction to aspirate fluid in an aspiration direction and to reverse direction of the aspirated fluid for pumping the aspirated fluid into a clot capture container. Circuits, systems, and methods can be used in general procedures for removing obstructive matters in any bodily lumen, such as to treat pulmonary embolism.

### CROSS-REFERENCES TO RELATED APPLICATIONS

The present application claims the benefit of and priority to U.S. Provisional Patent Application No. 63/794,251, filed Apr. 24, 2025, the content of which is hereby incorporated by reference in its entity.

### FIELD

The present inventions relate to systems and methods for treatment of clots, including intravascular removal of emboli and/or thrombi from a blood vessel of a patient. In particular, the present inventions relate to pull-push circuits integrable in clot extraction assemblies and used for clot extraction methodologies.

### BACKGROUND

Venous and arterial thromboses are blockages of blood vessels caused by clot, referred to as thrombus. A common form of venous thrombosis is deep vein thrombosis (DVT). DVT is a blood clot that forms in the deep veins, typically in the leg or pelvis. If a thrombus breaks off, or embolizes, and flows to the lungs to lodge there, it becomes a pulmonary embolism (PE), or a blood clot in the lungs. In other words, PE is a blockage of pulmonary arteries by a substance that has moved from elsewhere in the body through the bloodstream. While the etiology of most PE cases is a piece of a DVT that has traveled to the lungs, fatty embolus, bacterial masses, cancerous tumors, IV catheter clots, and other detritus can also cause pulmonary blockages. PE can be generally grouped into two categories, upstream and downstream obstructions. In upstream obstructions, that is in the main or large branch of the pulmonary arteries, an embolus can severely compromise total blood flow within the lungs. In downstream obstructions, that is in large to medium pulmonary artery branches, the embolus can prevent a significant portion of the lung from participating in the exchange of gases to the blood resulting in low blood oxygen and buildup of carbon dioxide.

A PE that originated from a thrombus can be described on a scale of acute to chronic. Clot ages from soft gel-like acute clot to fibrous firm chronic clot. Acute thrombus is newly clotted blood such as a clot less than one week old, which often is gel-like, relatively simple to break apart, and may dissolve using thrombolytics. As the thrombus ages, it can turn fibrous and can adhere to vessel walls. After a few (e.g., three) weeks, a thrombus generally is firm and organized, which is considered chronic. Chronic clot is difficult yet possible to aspirate but is highly resistant to dissolution using thrombolytics.

Surgical techniques have been used for embolectomy (removal of emboli) or thrombectomy (removal of thrombi). For example, a surgery may involve incising a blood vessel, followed by introducing a retrieval device to the location of the occlusion. The retrieval device can be deployed into, through, or around the clot and used to traverse the clot back to the point of incision to be removed by the physician. Although surgical techniques can be effective, surgery, by its nature, is invasive and harbors many risks including bleeding, infections, and further blood clots - not to mention prolonged anesthesia which can have its own complications. Moreover, the device used for removing the clot has a propensity of damaging the interior lining of the vessel as the device, carrying the clot, is withdrawn.

As an alternative to surgery, percutaneous methods such as balloon angioplasty can be used for reestablishing blood flow. Although balloon angioplasty has been the standard practice for treating vessel stenosis, it has proven to be ineffective for treating acute thromboembolisms and more so for removing occlusive clots. Also, balloon angioplasty may cause restenosis, which is the migration and proliferation of vascular smooth muscle cells within a blood vessel wall triggered by the impact of the balloon remodeling, leading to re-occlusion of the treatment site and the need for implanting a drug eluting stent. Another percutaneous technique involves placing a catheter near the clot and infusing streptokinase, urokinase, or other thrombolytic agents to dissolve the clot. Unfortunately, thrombolysis typically takes hours to days to be successful. Not to mention, thrombolytic agents can cause hemorrhage and in many patients the agents cannot be used at all. Thrombolysis is also ineffective at dissolving clots that have evolved to being partially or fully chronic.

These problems have led to the emergence of catheter based aspiration thrombectomy. Various aspiration devices currently exist in the market for performing thrombectomy and embolectomy. For example, FIG. 1 shows an aspiration catheter 2 to which a syringe 4 is coupled via a fluid control device 6. An aspiration tube 8 connects the fluid control device 6 to the catheter 2. The distal end of the aspiration catheter 2 is positioned in proximity to the clot. In use, a physician closes the fluid control device 6 and retracts the plunger of the syringe 4 to generate a vacuum in the barrel of the syringe 4. Once the physician has withdrawn the plunger by a desired amount, the physician can lock the plunger by rotating the plunger relative to the barrel, or otherwise the physician can instead hold the plunger in position relative to the barrel. The volume of the vacuum can be regulated and is commensurate to the degree to which the plunger is pulled back, to provide a desired level of suction upon opening of the fluid control device 6. To aspirate a clot, the physician can open the fluid control device 6 to release the vacuum stored in the barrel. Pre-charging or storing the vacuum before applying the vacuum through the catheter 2 generates a suction force that produces high fluid flow velocities of aspirated blood.

The drawbacks and disadvantages associated with this system are, however, numerous. The illustrated syringe system does not allow for reinfusion of the removed blood back into the patient to mitigate excessive blood loss from the procedure. Moreover, the syringe barrel has a finite volume and if the syringe does not have sufficient volume to contain the entirety of the clot along with the co-aspirated blood volume, the syringe needs to be replaced or reset, and the process repeated. In other words, if a first pass with a device does not completely capture all the clot material, which is likely, the fluid control device 6 must be closed, the syringe 4 removed and emptied or replaced with another syringe 4, and a vacuum reestablished by pulling back the plunger of the syringe 4, followed by another aspiration attempt. Additional resets can be made until the clot is removed or the physician otherwise ends the procedure. If several clots are present, numerous resets may be required. Additionally, as the syringe fills with blood and/or clot, the amount of vacuum pressure within the syringe decreases, leading to less effective aspiration as the syringe fills. A finite volume approach such as forming a vacuum using a syringe is further quite sensitive to air ingress. If the syringe and catheter are not well primed such that air remains in the syringe and/or catheter, a capability to form a vacuum may be significantly reduced. Finally, if the clot becomes stuck in the aspiration tube 8, the aspiration tube 8 needs to be removed from the patient in order to clear the clot outside of the patient’s vasculature. In order to clear the clot, the syringe 4 must be disconnected from the aspiration tube 8 and refilled with saline before reconnecting the syringe 4 to the aspiration tube 8 and forcing saline into the aspiration tube 8 to push out the clot. The aspiration tube 8 must be then reintroduced into the patient over a guidewire, through the heart, and into the pulmonary vasculature to remove additional clot. This is a non-trivial procedure, and crossing the heart can cause damage to the heart (and the device). Thus, the operation of such devices is cumbersome, its utility and applications are limited, and the duration of a procedure using such a device can become needlessly prolonged or early terminated to the detriment of the patient.

Accordingly, there has been an unmet need for new devices, systems, and methods for, inter alia, expedient and efficient removal of clots.

### SUMMARY

In accordance with one aspect, a circuit for removing a clot from a patient is provided. The circuit can comprise a reciprocal tube configured to be coupled to an aspiration catheter for suctioning fluid from a patient, and a pump connected to the reciprocal tube. The pump can be configured to apply suction to the aspiration catheter for aspirating the fluid in an aspiration direction along the reciprocal tube, and pump the aspirated fluid in a clot capture direction, reverse of the aspiration direction, along the reciprocal tube to a clot capture container. A controller can modify operation of the pump from a first mode of operation of aspirating the fluid and displacing the aspirated fluid in the aspiration direction along the reciprocal tube, to a second mode of operation of pumping the aspirated fluid and displacing the aspirated fluid in the clot capture direction along the reciprocal tube toward the container.

A first valve can be connected to the reciprocal tube downstream of the aspiration catheter, when flow is in the aspiration direction. The first valve can be configured to or is adjustable to prevent backflow of the aspirated fluid from the reciprocal tube to the catheter. The first valve can be configured to or is adjustable to prevent backflow of the aspirated fluid from the reciprocal tube to the catheter when the pump is not pumping.

The clot capture container can be connected in a flow path in the clot capture direction. The clot capture container can be positioned in the flow path to prevent or minimize flow attenuation between the pump and the aspiration catheter, and/or minimize air ingress into an aspiration side of the circuit when the clot capture container is opened to remove or inspect clot.

The clot capture container can be connected to the reciprocal tube by a clot capture line. The clot capture direction can extend along the clot capture line to the clot capture container for clot to be removed from the aspirated fluid.

A second valve can be connected to the clot capture line at an upstream position of the clot capture container. The second valve can be configured to or is adjustable to prevent backflow of the aspirated fluid from the clot capture line into the reciprocal tube.

A blood reservoir assembly can be connected to the clot capture container for receiving aspirated fluid. The operation of the pump in the second mode of operation can promote filtered aspirated fluid to be reinfused into the patient from the blood reservoir assembly.

A third valve can be positioned between the clot capture container and the blood reservoir assembly to prevent backflow of the aspirated fluid into the clot capture container.

The pump can be a peristaltic pump.

The pump can be connected to a saline reservoir by a saline line with an inner diameter greater than 1/8 inch. The circuit can be configured to allow the pump to purge the reciprocal tube with saline solution from the saline reservoir during the pumping of the aspirated fluid in the clot capture direction.

The circuit can be a closed circuit to allow de-airing of the reciprocal tube with saline solution by pumping saline solution from a saline reservoir connected to the pump through the reciprocal tube.

The circuit can additionally include a blood reservoir assembly connected to the clot capture container for receiving aspirated fluid and a filter positioned with the blood reservoir or downstream of the blood reservoir to remove clot or particulate from the aspirated blood.

In accordance with one aspect, a system for removing a clot from a patient is provided. The system can comprise a network of tubes for allowing blood and clot aspirated from a patient to move in an aspiration direction and a clot capture direction for depositing the clot in a clot capture container. The system can comprise a pump configured to independently control the flow rate of blood in the aspiration direction and the clot capture direction. The flow rate of the blood in the aspiration direction can be different than the flow rate of the blood in the clot capture direction. The clot capture direction can be in the opposite direction of the aspiration direction along at least a segment of the same tube. The aspiration direction can be from a catheter toward the pump along a segment of a tube connecting the catheter to the pump, and the clot capture direction can be in an opposite direction of the aspiration direction along at least a part of a same segment of the tube. The flow rate of the blood in the clot capture direction can be set to minimize hemolysis by reducing the velocity of blood delivered to a blood reservoir. The pump can be a peristaltic pump, the operation of which can be continuously switched between pulling and pushing fluid in the aspiration direction and clot capture direction, respectively.

In accordance with one aspect, a method of removing a clot from a patient is provided. The method can comprise positioning a catheter at a target site of the patient, aspirating fluid from the catheter into a reciprocal tube, and reversing the flow of aspirated fluid flowing along the reciprocal tube to reroute the aspirated fluid toward a clot capture container for capturing a clot. The method can also comprise continuously repeated aspirating fluid and reversing the flow of the aspirated fluid to remove more clot from the patient. The flow of aspirated fluid can be reversed prior to the aspirated fluid reaching a pump used to suction the aspirated fluid from a catheter. The pump can be further used to pump the aspirated fluid into the clot capture container in a clot capture direction.

The method can further include pumping aspirated fluid through the clot capture container and into a blood reservoir assembly in which the aspirated fluid can be filtered for reinfusion into the patient.

The method can further include suctioning saline solution from a saline reservoir when the flow of aspirated fluid is reversed to purge the aspirated fluid from the reciprocal tube into the clot capture container.

In accordance with one aspect, a clot extraction system is provided. The system can comprise a first tube having a distal end coupled to a catheter, a clot capture container coupled by a second tube to the first tube, and a pump connected to a proximal end of the first tube and configured to apply negative pressure to the first tube to aspirate a clot from the patient through the catheter and to apply positive pressure to the first tube to transport the clot to the clot capture container through the second tube. The negative pressure can be, for example, from about negative 10 psi to about negative 14.7 psi to the first tube.

A first valve can be connected to the first tube downstream of the catheter to prevent backflow of fluid from the first tube into the catheter. A second valve can be connected to the second tube to prevent backflow of fluid from the second tube into the first tube.

A controller can be provided for controlling the operation of the pump from a first mode of operation of applying negative pressure to the first tube to aspirate the clot from the patient through the catheter to a second mode of operation of applying no pressure or positive pressure to the first tube. The positive pressure can be applied to the first tube to transport the clot to the clot capture container through the second tube.

A saline reservoir can be connected to the pump for supplying a saline solution to the first tube. A blood reservoir can be connected to the clot capture container for receiving fluid from the clot capture container. A filter can be included to filter blood in the blood reservoir. A third tube can be included to reinfuse the filtered blood into the patient.

At least one sensor can be positioned along the first tube and configured to detect a presence of aspirated fluid and communicate a signal to halt or reverse operation of the pump prior to the aspirated fluid entering the pump.

At least one sensor can be positioned along the first tube. The at least one sensor can be configured to detect a presence or position of the aspirated fluid in the first tube and communicate a signal to a controller. The controller can be configured to modify operation of the pump from a first mode of operation to a second mode of operation in response to the signal from the sensor indicating that the aspirated fluid is approaching the pump. The at least one sensor can comprise a first sensor positioned to determine a quantity of saline in the first tube, and a second sensor positioned at a selected distance from the pump to detect when aspirated fluid is approaching the pump.

In accordance with another aspect, a method of treating pulmonary embolism with the circuits, systems, and methods is provided.

### BRIEF DESCRIPTION OF THE DRAWINGS

FIG. 1 is a prior art catheter-syringe device used to aspirate clots.

FIG. 2 is a schematic diagram of an embodiment of a clot extraction system of the present inventions, also illustrating an embodiment of the pull-push circuitry component of the system.

FIG. 3 is a flow diagram illustrating a method of using a clot extraction system of the present inventions in accordance to one embodiment.

FIG. 4 is an example of a peristaltic roller pump that can be used with the embodiments of the present inventions.

FIGS. 5A, 5B, and 5C, are examples of valves that can be used with the embodiment of the present inventions.

FIG. 6 is a schematic diagram of another embodiment of a clot extraction system of the present inventions.

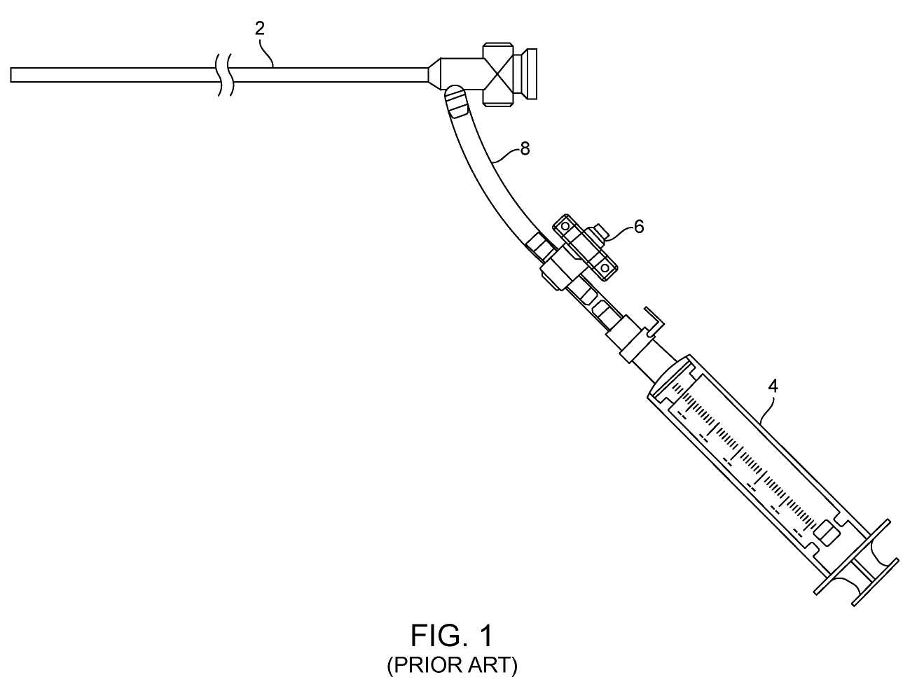

### DETAILED DESCRIPTION

The present inventions relate to systems and methods for treatment of clots, including intravascular removal of emboli and/or thrombi from a blood vessel of a patient (human or animal). In particular, the present inventions relate to pull-push circuits integrable in clot extraction assemblies and used for clot extraction methodologies. Although the embodiments are described with respect to circuits, systems, and methods for removing a clot or otherwise treating pulmonary embolism, other applications are within the scope of the claims, including intravascular procedures for treating cerebral embolism, intravascular procedures for treating deep vein thrombosis (DVT), intravascular procedures other than the treatment of emboli, as well as general procedures for removing obstructive matters in any bodily lumen.

### Terminology

A few terms that may be used herein are described below. These descriptions are for convenience only and are not limiting.

The term “vessel” refers to any vein or artery, including vessels in a venous branch or an arterial branch.

The term “clot” refers to all types of obstructive matters. Examples of obstructive matters which are intended to be extracted by the circuits, systems, and methods of the present inventions include, but are not limited to, attached and detached solid and semi-solid masses (e.g., blood masses or blood cell and platelet coagulations); plaque (e.g., cholesterol, cellular waste products, calcium, fibrin, and protein and amyloid deposits); fibrous material; bacterial clumps; fat globules; benign or cancerous tumor fragments; and foreign materials and objects (e.g., pieces of surgical sponges and metallic or plastic fragments). Bacterial clump can be caused by an infective endocarditis bacterium, such as staphylococcus aureus, enterococcus faecalis, or enterococcus faecium associated with heart valve leaflets. The embodiments of the present inventions are useful for removal of all types of clot, including a long clot (e.g., over 1 cm in length, or over 10 cm in length) from a vessel.

The term “thrombus” (or the plural “thrombi”) refers to clot formed in situ within the vascular system that impedes or obstructs blood flow within a vessel. A thrombus is fixed within a vessel but has the potential of breaking off and forming an embolus which then can travel through the bloodstream and lodge in the vessel or another vessel which can cause an obstruction or blockage of the vessel.

The term “embolus” (or the plural “emboli”) refers to clot which has traveled in the bloodstream and lodged in a vessel, which can cause an obstruction or blockage of the vessel. The term “pulmonary embolism” refers to the situation in which an embolus obstructs or blocks a pulmonary artery of any size. This blockage prevents blood from reaching areas of the lungs, leading to oxygen deprivation and potential tissue damage. The term “coronary embolism” refers to a situation in which an embolus obstructs or blocks a coronary artery of any size. This blockage prevents blood from reaching areas of the heart, which can lead to myocardial dysfunction and myocardial infarction.

The term “hemolysis” refers to the destruction of red blood cells.

The term “fluid” refers to a gas or a liquid or a combination of gas and liquid, and the fluid can have materials dispersed within. For example, fluid is intended to include any bodily fluid or a semi-solid biofluid, including blood, with or without clot. Fluid is also intended to include saline, or a mixture of saline and a bodily fluid/semi-solid biofluid. Thus, for example, aspirated fluid can include blood, with or without a clot, or blood to which saline solution has been added, with or without a clot. Fluid is also intended to include clot-less filtered bodily fluid, with or without an active agent, such as an anticoagulant and/or a thrombolytic agent. The definition of fluid is apparent based on the context in which it is used.

The term “line” when used with respect to fluid transfer refers to a tube, lumen, channel, duct, or other passageway through which fluid can flow.

One example of a line can be a catheter. An example of a catheter is an aspiration catheter used to remove fluid via suction from a target location of a patient. Aspiration catheters are generally flexible and of sufficient length to reach the target location. An aspiration catheter can have one or more lumens. A vacuum lumen can be formed in or extended through a length of an aspiration catheter. An inner diameter of a vacuum lumen should be sufficiently sized to preserve an applied vacuum pressure while allowing for removal of clots of various sizes. An aspiration catheter can be used by itself or in conjunction with a catheter system having a working channel in which the aspiration catheter can be inserted and navigated to reach a target site or in which the working channel defines the aspiration catheter. The catheter system can also include other working channels configured to receive guidewires and/or other interventional devices and instrumentations.

The term “valve” refers to a mechanical or electromechanical device for controlling the direction and/or the degree of flow of fluid through a line, tubing, lumen, connector, fitting, duct, reservoir, or container. A valve can be, for example, configured to allow or limit fluid movement in one or more directions (e.g., one-way, two-way, or n-way valve). A valve can be controlled manually (e.g., by manipulation of a mechanical feature) or automatically (e.g., transmission of an electrical signal via a controller), or by a state of the fluid (e.g., pressure or temperature).

Examples of valves include, but are not limited to, pinch valves (e.g., controlling the flow of fluids by "pinching" a flexible rubber sleeve inside the valve body), timed valves (e.g., controlling the valve opening and closing according to a schedule or period), pressure differential or differential pressure control valves (e.g., a pressure difference between two points in a system causes the valve to naturally open, or causes a controller to automatically adjust valve opening based on a sensed pressure differential or a pump state), check valves, and hemostatic valves.

A check valve, as is known in the valve art, is a type of two-port valve – that is, it has two openings, one for a fluid to enter and the other for the fluid to exit. Check valves can work autonomously or can be controlled by an operator. A cracking pressure in a check valve is a minimum differential pressure between inlet and outlet at which the valve will begin to open. Cracking pressure is also known as unseating head pressure or opening pressure. Reseal pressure refers to the minimum pressure differential between the inlet and outlet of the valve during the closing process of the check valve, at which there is no observable leak rate. Reseal pressure is also known as sealing pressure or closing pressure. The check valve can be designed for and, therefore, can be specified for a specific cracking and reseal pressure, which for the purpose of the present inventions can be associated with transportation of fluid (e.g., blood and/or clot) in the stated directions. The types of check valves that can be employed with the embodiments of the present invention (see, for example, FIGS. 5A-5C discussed below) can include, but are not limited to a ball check valve, in which the closing member or the movable part to block the flow is a ball or a spring loaded ball, a diaphragm check valve that uses a flexing rubber diaphragm in which the pressure differential between the upstream side and the downstream side causes the diaphragm to open, a swing check valve or tilting disc check valve that has a movable disc to block the flow (the disk swings on a hinge or trunnion to block reverse flow or to allow forward flow), a stop-check valve that is a check valve with override control to stop flow regardless of flow direction or pressure, a lift-check valve in which a disc, also referred to as a lift, is elevated by higher pressure of inlet or upstream fluid to allow flow to the outlet or downstream side (when the pressure drops, gravity or higher downstream pressure will cause the disc to lower, shutting the valve to stop reverse flow), and an in-line check valve that is a check valve similar to the lift check valve but with a spring (an adequate crack pressure is needed to overcome the spring force and when the pressure going through the valve goes below the resealing pressure, the spring will close the valve to prevent back-flow in the process). A resealing pressure may be lower than the cracking pressure. A check valve may, in some cases, be assisted or controlled mechanically, pneumatically, or electrically. In some cases, a check valve may be assisted to reseal at a pressure lower than a resealing pressure would be absent the assistance.

A two-way valve can be controlled to function as a check valve via control. For example, a state of a two-way valve can be linked to a differential pressure measurement. The valve is toggled based on the differential pressure measurement to ensure one way flow. Alternatively, a two-way valve state can be linked to a fluid actuator state, such as a syringe plunger state or pump direction.

A hemostatic valve, for example, may prevent fluid from flowing around an instrument or tube when the instrument or tube is passed through the hemostatic valve, and fully prevent fluid flow through the valve when no instrument or tube is passed through the hemostatic valve.

A hemostatic valve placed at a proximal segment of a catheter or catheter system can allow for insertion of the aspiration catheter into a working channel of a catheter system while maintaining hemostasis during a procedure such as a clot removal procedure.

The term “pump” refers to a device used to cause or encourage a movement of fluid through a structure. For example, a pump can refer to a device configured to create suction or pull (e.g., negative pressure) and/or discharge or push (e.g., positive pressure). Such pump can create pull or push within a tube and/or create suction at an ingress and discharge at an egress of the tube or the pump.

A pump can be a syringe pump. A syringe pump can be operated manually or operated by an actuating machine with a motor to manipulate the syringe plunger in a controlled rate to extract or deliver the fluid, blood, and/or clot. Syringe pumps come in standard configurations, or continuous configurations such as including two syringes. A syringe pump system may include automated or controlled valves (e.g., check valves, two-way valves, etc.) to be able to draw from one source and dispense to another source.

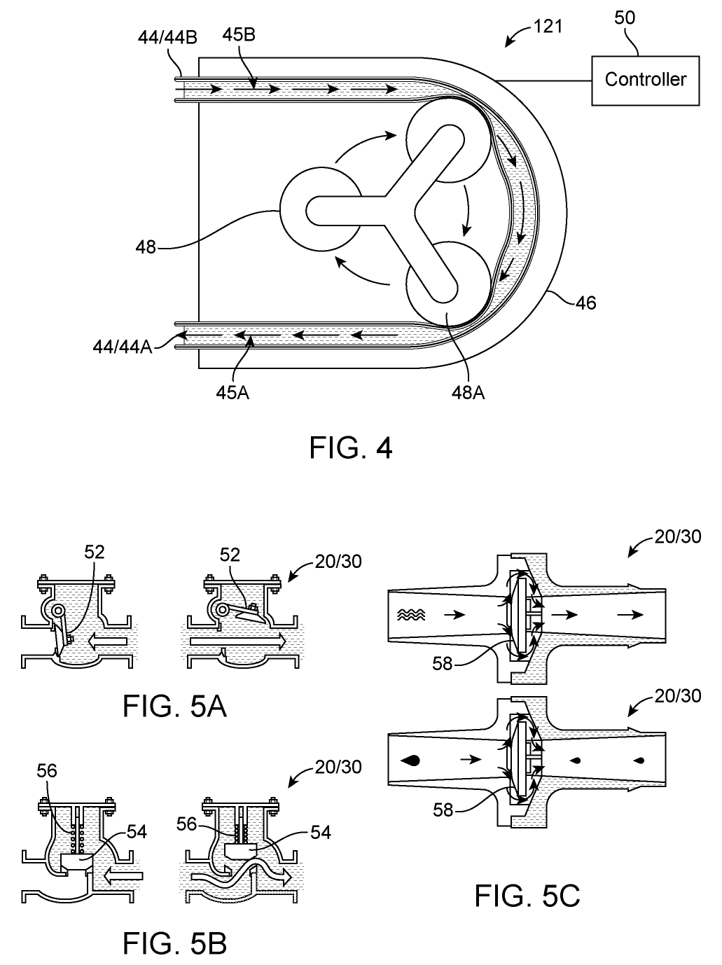

A pump can be a peristaltic pump. A peristaltic pump is also commonly known as a roller pump, and is a type of displacement pump. It can be used for removing and positively displacing blood or saline. In an embodiment, blood can be contained in a flexible tube, a segment of which is fitted inside a circular pump casing, as will be described in more detail below with reference to FIG. 4. Examples of roller peristaltic pumps include rotary motion peristaltic pumps and linear peristaltic pumps. In a rotary motion peristaltic pump, a rotor has a number of wipers or rollers attached to its external circumference, the rotor rotates around an axis, and the wipers or rollers compress the flexible tube as the rotor rotates. In a linear peristaltic pump, a set of translating or rotating wipers or rollers are individually moved to concurrently or sequentially compress a flexible tube. As the part of the tube under compression closes, fluid is forced to move through the tube. As the tube reopens to its natural state after the compression is released (e.g., the rollers pass in a rotary peristaltic pump), more fluid is drawn into the tube (e.g., behind the rollers as they pass).

A pump can be a diaphragm pump. A pump can be hydraulically coupled to an exterior of an expandable vessel (e.g., tube, bag, etc.) with check valves at an entrance and exit to the expandable vessel, where oscillating pressure within the hydraulic coupling moves fluid into and out of the expandable vessel.

A pump can be another type of pump other than the ones mentioned above, or a combination of pumps and/or pump types. Aspects of a suitable pump for the embodiments of this disclosure can include a capability for high lift, a capability for high flow, a fluid path that does not generate debris sufficient to prohibit blood return, a sterilizable fluid path, etc.

The term “clot capture container” refers to a container which collects fluid and optionally separates the bulk of solid or semi-solid materials (e.g., clot) from liquid (e.g., blood) in the fluid. The container has an inlet through which fluid enters; the fluid can flow into or through the container and can optionally deposit clot in a clot-separation portion of the container. The container can have an outlet configurable to be in communication with other components. In an embodiment, the clot capture container can become vacuum sealed by fluid flowing through the clot capture container under vacuum. In an embodiment, a clot capture container can include a transparent window or removable cover to allow a physician to observe whether a clot has been captured and the amount of clot captured without having the need to open the container which can cause a breach of a sterile environment.

The term “aspiration connector” refers to a device accessory that allows for the attachment of a suction line or syringe to a medical instrument, such as an aspiration catheter, enabling the suctioning of fluid during a procedure. Aspiration connectors are designed to create a secure and sealed connection between a suction source and a catheter, allowing for controlled aspiration of fluid.

The term “communication” means that a fluid, a solid matter, or a signal (e.g., signal from a sensor) can flow or travel from one component of a system to one or more components of the system or another system. The flow or travel can be direct or indirect. Direct flow or travel means that the fluid flows, the solid matter transfers, or the signal travels from component A to component B without any intermediate or intervening components being between components A and B through which the fluid flows, the solid matter transfers, or the signal travels. Indirect flow means that the fluid flows, the solid matter transfers, or the signal travels from component A to component B with one or more intermediate or intervening components, such as component C and/or D, being in between components A and B through which the fluid also flows, solid matter also transfers, or the signal also travels. With respect to the specification and claims, all communication can be direct or indirect unless otherwise specified.

The terms “coupled,” “engaged,” “attached,” and “connected” are terms that are used interchangeably and are intended to mean the same. These terms can mean physically joined. Physically joined can mean that component A is permanently secured or fastened (e.g., via a weld, solder, adhesive, screw, etc.) to component B, or detachably joined such that that component A can be detached or disconnected from component B (e.g., friction fitted connection or by a mechanical coupling feature). Disconnecting or detaching can be performed by a user or a maintenance technician.

The terms “coupled,” “engaged,” “attached,” and “connected” can also mean communication as defined herein – that is, for example, the coupling, engagement, attachment, connection, or communication can be direct or indirect. Direct means that component A is in contact with or touching component B without any intermediary or intervening components being placed between components A and B (e.g., part-to-part contact or fluid/solid/signal flows/transfers/travels from component A to component B without passing through any other component). Indirect means that an intermediary or intervening component is placed in between components A and B (e.g., fluid/solid/signal flows/transfers/travels from component A to component B by passing through component C). With respect to the specification and claims, all coupling, engagement, attachment, connection, or communication are intended be direct or indirect and permanent or detachable unless otherwise specified. For example, “a pump connected to a catheter” can mean that the pump is in communication with and detachable from the catheter to allow body fluids to be aspirated by the pump through the catheter and other components, such as tubes, valves and connectors that can be optionally attached along the flow path of the aspirated fluid between the catheter and the pump.

The term “sealed” or “sealed engagement” means that fluid is prevented from entering, discharging, or unintentionally leaking under normal or intended operating and use conditions.

The terms “proximal” and “distal” are terms used to reference a position of a component relative to another component, an operator, or a patient. Proximal refers to relatively near the component, operator, or patient and distal refers to relatively distant from the component, operator, or patient.

The singular forms “a,” “an,” and “the” are intended to include the plural forms and plural is intended to include singular, unless the context clearly indicates otherwise. Thus, for example, “clot” can include multiple clots.

The terms “first, “second,” and the like are used simply to distinguish one feature or element from another feature or element and are not intended to define any order of the components. Thus, a first feature or element discussed herein could be termed a second feature or element, and similarly, a second feature or element discussed herein could be termed a first feature or element. For example, “a first valve” and “a second valve” does not connote any particular order of the positioning of the valves but is merely used to distinguish one valve over another valve. Moreover, when a claim refers to a “first component” (e.g., “first valve”), this should not be construed to imply or exclude the presence of a second component (e.g., “second valve”) within the scope of the same claim.

The terms “about,” “approximately,” “substantially,” and “generally” are used to represent an amount, value, or degree that is close to the stated amount while still performing the same desired function or achieving the same result. The terms can refer to an amount, value, or degree that is within less than or equal to 10% of the stated amount, preferably less than or equal to 5% of the stated amount, and most preferably less than or equal to 1% of the stated amount.

The term “and/or” includes one or any combination of one or more of the associated listed items.

The terms “in an embodiment,” “in one embodiment,” or “in another embodiment” means “in one or more embodiments.”

The term “comprise” is interchangeable with the term “include,” such that “comprising” corresponds to “including,” “comprises” or “comprised of” corresponds to “includes,” “is comprised in” corresponds to “is included as part of,” and so forth. A list of items following “comprising,” “comprises,” “comprised of,” or the like is therefore non-limiting, and the list may additionally include other items not listed.

The terms “e.g.,” “such as,” “for example,” “and so forth,” and “etc.” mean that what follows the “e.g.,” “such as,” or “for example” or what precedes the “etc.” or “and so forth,” is a list of examples and there may be other items that could also serve as examples but are not listed.

Standard measurement units and their abbreviations may be used herein, such as “mm” for millimeter(s), “cm” for centimeter(s), “cc” for cubic centimeters, “mL” for milliliter(s), and so forth.

### Examples of Embodiments

The systems of the present inventions comprise a pump (e.g., a peristaltic pump) that is in communication with an aspiration catheter via a reciprocal tube. A controller can operate modes of the pump to direct movement of aspirated fluid from the catheter into the reciprocal tube (in an aspiration direction) and direct movement of fluid from the reciprocal tube toward a clot capture container (in a clot capture direction) or other container. The bi-directional, or back-and-forth, flow of the aspirated fluid through at least a segment of the reciprocal tube defines the “pull-push” characteristic of the present inventions – that is, the fluid is both pulled into and pushed out of at least a section of the reciprocal tube by the pump. The flow rate of the fluid in the aspiration direction can be (or configured to be) different than the flow rate of the fluid in the clot capture direction. In an embodiment, the fluid flow rate is higher in the aspiration direction than in the clot capture direction, which would allow for a more aggressive application of suction in the aspiration direction which in turn may allow for improved retrieval of a clot, and a gentler application of flow in the clot capture direction should the system be configured to reinfuse aspirated blood into the patient. The system can comprise a valve (e.g., check valve) that can allow for the back-and-forth, or “pull-push,” movement of the aspirated fluid in the reciprocal tube to be directed in the aspiration direction or the clot capture direction (or other direction) as applicable.

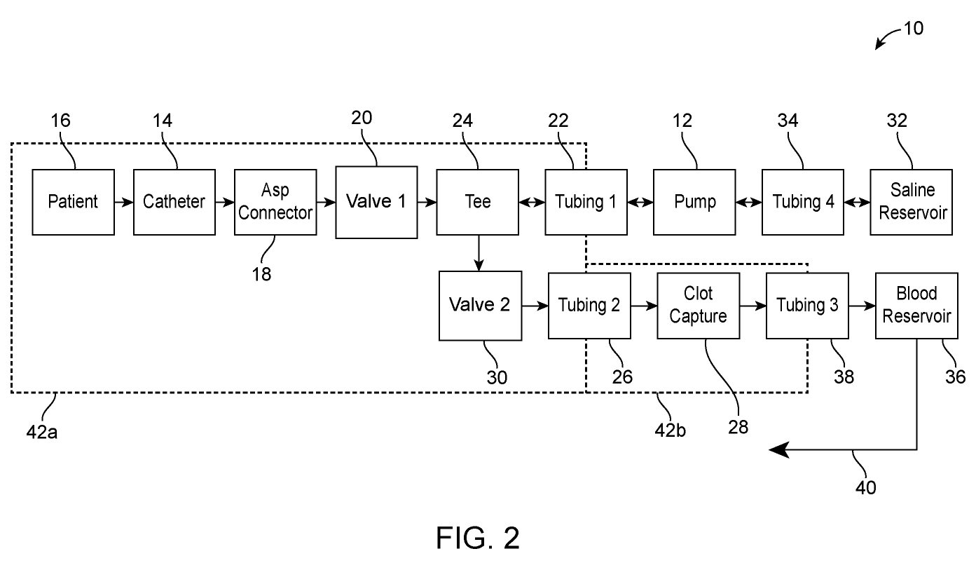

Referring to FIG. 2, a schematic of an embodiment of an aspiration system 10 of the present inventions is illustrated. In FIG. 2, distal refers to toward the patient and proximal refers to away from the patient and toward an aspiration source. An arrow pointing in one direction means that the system 10 is configured to allow fluid to flow only in the direction of that arrow. An arrow pointing in both directions means that the system 10 is configured to allow fluid to flow in both directions. The system 10 comprises a pump 12 (e.g., a peristaltic pump) in communication with an aspiration catheter or a catheter system 14 (hereinafter referred to as “catheter 14”), which can be percutaneously inserted into a patient 16 for extraction of a clot. An aspiration connector 18 can couple a first valve 20 (e.g., a check valve) to the catheter 14. The first valve 20 allows for the suctioned fluid (e.g., blood and/or clot) to flow from the catheter 14 towards the pump 12, but prevents reverse flow of fluid, including the aspirated fluid, back through the first valve 20 and toward or through the catheter 14.

In an embodiment using a check valve as the first valve 20 and assuming a patient blood pressure of about 2.3 psi, a cracking pressure of the first valve 20 of, for example, greater than about 3 psi can prevent the patient’s blood pressure from opening the first valve 20. Higher cracking pressures may be used, such as greater than 5 psi, or greater than 10 psi, or up to the maximum vacuum or the pump, and so forth. The higher the cracking pressure of a check valve, the higher a vacuum pressure will need to be on the proximal side of the valve before the valve opens (e.g., continuous vacuum or a build-up of vacuum pressure generated at the proximal side of the valve before the valve opens for aspirated fluid flow). For example, for a check valve with a cracking pressure of 13 psi, a vacuum pressure of more than 10 psi may need to be generated for flow to occur (which takes into account the assumed 2.3 psi generated by patient blood pressure). A check valve may remain open once opened until a resealing pressure is reached. In an embodiment, a resealing pressure is lower than the cracking pressure. In an embodiment, the first valve 20 can be a user actuated valve, the opening of which can be synchronized with the pump 12 being turned on for aspiration.

In an embodiment, as flow begins a differential pressure will decrease. Some check valves will begin to close as differential pressure decreases, and flow will reduce until the opening and closing pressure are in equilibrium; this would result in a no flow state when closed to a higher flow state when first opened and an intermediate decreasing flow state until flow is inhibited. Some check valves might open at a high cracking pressure and not begin to close until well below the cracking pressure and, once the valve begins to close it will relatively quickly close completely; this would result in a no flow state when closed to a higher flow state while opened until flow is inhibited (without an intermediate decreasing flow state or with a relatively very short intermediate decreasing flow state).

System 10 further includes a first line 22, which may be referred to as a reciprocal tube 22, extending between the first valve 20 and the pump 12. The reciprocal tube 22 can be a large diameter flexible tube, having an inner diameter of about 5 millimeters (mm) to about 13 mm. A large diameter tube provides a more efficient vacuum application, lowers blood flow velocity through the tube which can reduce hemolysis of the blood, and minimizes a possibility of a clot becoming lodged in the tube.

System 10 further includes a tee connector 24, which may be referred to as a fitting 24, coupled on a proximal side of the first valve 20 and a clot capture line 26 coupled to the fitting 24. An optional clot capture container 28 for receiving fluid having a clot can be coupled to the clot capture line 26. Placement of the clot capture container 28 away from the fluid path between the patient 16 and the pump 12 avoids having flow through the clot capture container 28 dampen a vacuum communication between the pump 12 and the catheter 14 during aspiration. A second valve 30 (e.g., a check valve) can be coupled downstream of the fitting 24 to prevent fluid from backflowing from the clot capture line 26 and/or clot capture container 28 into the reciprocal tube 22.

In operation, the pump 12 may cause a vacuum to be created in the catheter 14 such that fluid may be aspirated from the patient 16 through a flow path between the catheter 14 and the pump 12 and into the reciprocal tube 22. When the reciprocal tube 22 is filled, the pump 12 may then cause fluid flow to be reversed within the reciprocal tube 22; however, because the first valve 20 is configured to block reverse flow through the catheter 14 into the patient 16, fluid from the catheter 14 flows instead through the fitting 24 in the clot capture direction (e.g., into the clot capture line 26). Fluid then may flow through the clot capture container 28.

System 10 further includes a saline reservoir 32 connected to the pump 12 via a saline line 34. Saline solution can be used to purge the reciprocal tube 22 during the displacement of fluid from the reciprocal tube 22 in the clot capture direction. In other words, when the pump 12 pumps the fluid from the reciprocal tube 22 in the clot capture direction, it contemporaneously draws saline solution from the saline reservoir 32 to purge the reciprocal tube 22. Saline solution can also be used to remove air from the closed circuit of FIG. 2. The pump 12 can pull the saline solution from the saline reservoir 32 and through the saline line 34, and push the saline solution through the reciprocal tube 22, the fitting 24, and the clot capture line 26 to purge air. Thus, any needed de-airing of the system 10, more particularly between the pump 12 and the catheter 14, is simplified and the need to connect and disconnect components to remove air is eliminated. Saline solution can also be flushed into the clot capture container 28 to separate blood from clot or clear the clot capture container 28. Flushing of the clot capture container 28 with the saline solution can allow a physician to be able to see clot that has been captured that otherwise would not have been able to be noticed through dense and opaque biofluids.

In an embodiment, the saline line 34 is large enough that saline can be pumped from the reservoir at high speed. In an embodiment, the saline line 34 has an inner diameter of greater than about 3 mm (1/8 inch), such as greater than 6 mm (1/4 inch).

In an embodiment, the clot capture container 28 can be coupled to a blood reservoir 36 by a blood reservoir line 38. The blood reservoir 36 can collect blood received from the clot capture container 28. In an embodiment, the blood reservoir 36 can filter the blood.

The amount of saline solution that is introduced into the closed circuit of FIG. 2 from the saline reservoir 32 that is sufficient to move a bolus of aspirated fluid from the reciprocal tube 22 into the clot capture line 26 can be predetermined, and metered or measured during flow, to avoid or minimize saline from the saline reservoir 32 flowing into the blood reservoir 36 and diluting the blood in the blood reservoir 36.

Blood from the blood reservoir 36 can be used to reinfuse the patient’s blood into the patient 16 through a recirculation line 40.

The operation of the pump 12 can be controlled so that the fluid flow rate is higher in the aspiration direction than in the clot capture direction, which would allow for a more aggressive application of suction sufficient for retrieval of a clot and a gentler flow in the clot capture direction where high flow rate is not needed.

Hemolysis can be reduced by the action of system 10 as compared to other aspiration systems. One way that hemolysis can be reduced is by the positioning the clot capture container 28 outside of the aspiration flow path. If a clot chamber (e.g., the clot capture container 28) was positioned in the aspiration flow path, fluid aspirated from the patient 16 at relatively high velocity could impact on interior surfaces of the clot chamber at the relatively high velocity and cause hemolysis. Instead, the aspirated fluid is met with saline solution within the reciprocal tube 22 which can minimize hemolysis. Another way that hemolysis can be reduced is by moving a bolus of aspirated fluid at a relatively low flow rate from the reciprocal tube 22 in the clot capture direction, so that when the bolus reaches the clot capture container 28 or the blood reservoir 36 it will not impact walls at a high velocity and thus hemolysis may be reduced.

A procedure using the aspirated system 10 of FIG. 2 may be performed at least in part within a sterile environment (e.g., in an operating room). It can be difficult to maintain a sterile environment and thus it may be preferable to minimize an area demarcated as a sterile environment. As such, it may be preferable to minimize a footprint of components of the aspirated system 10 which must be positioned within a sterile area. A boxed outline 42 in FIG. 2 demarcates an example of how components of the aspiration system 10 may be included in or excluded from a sterile area or field. In the example of FIG. 2, the sterile field is shown separated into a boxed outline 42a and a boxed outline 42b for ease of discussion of FIG. 2. In an actual procedure environment, the sterile field size and contour (and number of sterile fields) will be as defined by the medical staff performing the procedure, and the selection of components to be placed within the sterile field will be as defined by the medical staff performing the procedure. At a minimum, it is expected that the catheter 14 would be within the sterile field, and preferably additional components of system 10 also would be within the sterile field. If so desired by the medical staff, the entirety of system 10 could be positioned within the sterile field.

In the example of FIG. 2, the boxed outline 42a includes the catheter 14, the aspiration connector 18, the first valve 20, the fitting 24, at least a portion of the reciprocal tube 22, the second valve 30, and at least a portion of the clot capture line 26; the boxed outline 42b includes the clot capture container 28 and at least a portion of the blood reservoir line 38. In an embodiment, the components in the boxed outline 42a may be requested by the manufacturer to be positioned within a sterile field. In an embodiment, the components in the boxed outline 42a and the components in the boxed outline 42b may be requested by the manufacturer to be positioned within a sterile field.

By placing the clot capture container 28 within a sterile environment, the medical staff can readily assess if clot has been aspirated and the volume of clot removed without breaching or compromising standard sterility protocols. To provide visibility, portions of, or the entirety of, the reciprocal tube 22 and/or the clot capture line 26 can be made of a transparent material. In an embodiment, the clot capture line 26 can have a cover made of transparent material. In an embodiment, the clot capture line 26 can have a removable cover. Transparent materials and removable covers can allow the medical staff to see and assess aspirated clots.

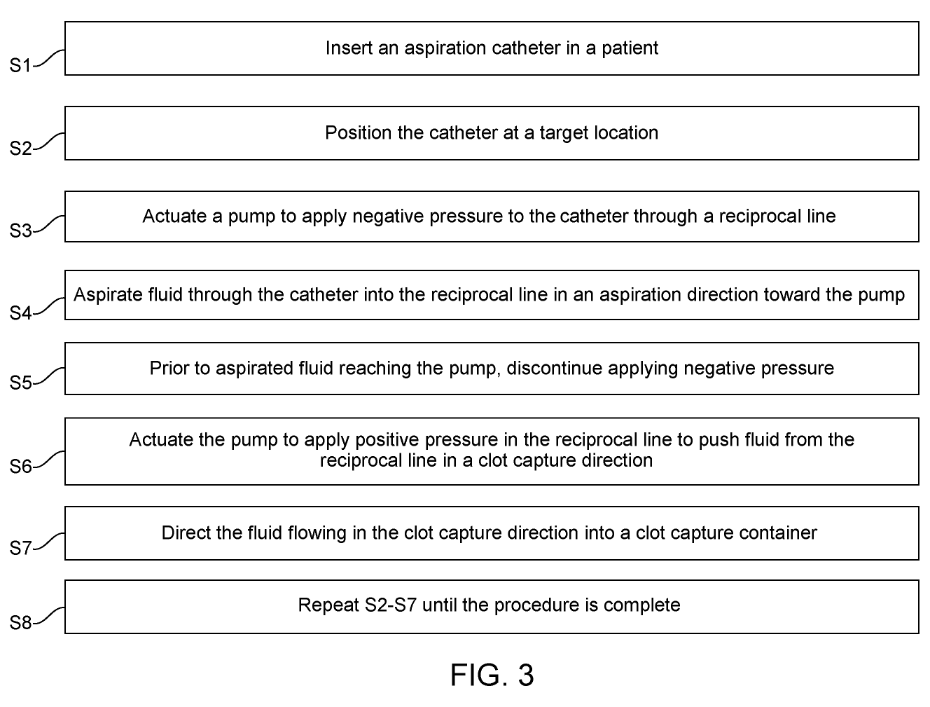

FIG. 3 is a flow diagram of an example of an embodiment for performing clot removal using an aspiration system such as system 10. At S1, a catheter (e.g., the catheter 14) is entered into the body of a patient (e.g., percutaneously), navigated through the patient (e.g., through a vessel) and at S2 positioned at a target site (e.g., a pulmonary artery). At S3, a pump (e.g., the pump 12) is activated to apply negative pressure (suction), for example from about negative 10 psi to about negative 14.7 psi, to aspirate fluid from the patient into a reciprocal tube (e.g., reciprocal tube 22). At S4, fluid (e.g., blood and clot) is aspirated in an aspiration direction (e.g., from the first valve 20, through the reciprocal tube 22, and towards the pump 12.) In an embodiment, about 10 cc to about 60 cc of fluid (or more) can be aspirated from the patient and drawn into the reciprocal tube in the aspiration direction. At S5, before the aspirated fluid flowing through the reciprocal tube reaches the pump, the pump action is modified to discontinue negative pressure. At S6, operation of the pump is again modified to apply positive pressure into the reciprocal tube and urge the aspirated fluid in the reciprocal tube in a clot capture direction (e.g., from the reciprocal tube 22 through the second valve 30, through the clot capture line 26, and into clot capture container 28.) A saline reservoir (e.g., the saline reservoir 32) can act as a purge tank should the aspirated fluid inadvertently by-pass the pump. As aspirated fluid is driven by positive pressure in the clot capture direction, fluid from the saline reservoir is drawn into the reciprocal tube to backfill the reciprocal tube where the aspirated fluid was held and thereby avoid introducing air into the reciprocal tube. A valve (e.g., the first valve 20) can operate to prevent flow of fluid from the reciprocal tube to the catheter while the fluid is pushed in the clot capture direction.

In an embodiment, about 10 cc to about 60 cc of fluid (or more) aspirated from the patient and drawn into the reciprocal tube at S3, and any added saline solution, is pushed in the clot capture direction at S7, into a clot capture container (e.g., the clot capture container 28,) and optionally through the clot capture container and into a blood reservoir (e.g., the blood reservoir 36.) The aspirated blood and clot traverse a tee or other fitting (e.g., the fitting 24,) traveling through the clot capture line and into the clot capture container, where the collection can optionally be observed and assessed by the physician. The second valve, placed upstream of the clot capture container, prevents backflow of blood and saline into the reciprocal tube. In an embodiment, aspirated fluid can be pumped through the clot capture container and into the blood reservoir, where it can be treated, filtered, and optionally reintroduced (reinfused) into the patient.

To prevent or minimize hemolysis, the aspirated fluid is preferably not allowed to traverse the pump; therefore, the pump can be controlled to discontinue aspiration (i.e., discontinue generating a negative pressure) when the aspirated fluid has traversed about half to three quarters of the length of the reciprocal tube. The first valve prevents the aspirated fluid and saline that is forced in the clot capture direction from flowing back into the catheter.

At S8, the pull-push operation described by S1-S6, S1-S7, S2-S6, or S2-S7, or another sequence of operations, can be repeated as desired, and as often as desired, until the procedure is ended (e.g., a physician ends the procedure when an arterial blood pressure of the patient falls to or below a threshold pressure acceptable to the physician, a total procedure time reaches or exceeds a threshold time set by the physician, a condition of the patient dictates that it would be preferred to end the procedure, or there are no additional clots that the physician strives to aspirate.)

The pull-push cycling of aspirated fluid, and optionally saline solution, can also be performed to remove any lodged clots from the reciprocal tube or from the flow path in the clot capture direction that may constrain optimum performance of the circuit.

Notably, the process described by FIG. 3 does not require creating a stored volume of vacuum pressure prior to or during the procedure.

FIG. 4 illustrates an example of a peristaltic roller pump 121 that can be used as the pump 12. A flexible tube 44 is fitted inside a circular pump casing 46. Roller peristaltic pumps work through rotary motion of wipers or rollers 48 applying and releasing pressure on the flexible tube 44. The rollers 48 compress the flexible tube 44 as the rollers 48 rotate. As part of the tube 44 is compressed, fluid ahead of the roller 48 (e.g., fluid 45A in tube 44 ahead of roller 48A) is urged to move through the tube 44 by the pressure induced in the tube 44 by the roller 48. After the rollers 48 pass, the tube 44 opens to its natural state and more fluid is drawn into the tube 44 (e.g., fluid 45B in the tube 44.) A portion or extension 44A of the tube 44 can be coupled to the saline line 34 and a counterpart portion or extension 44B can be coupled to the reciprocal tube 22. FIG. 4 illustrates, by the arrows in the tube 44, a flow direction corresponding to the clot capture direction (i.e., from the extension 44B towards the extension 44A).

A controller 50 can control a mode of operation of the pump 121 (along with any valves). In a first or pull mode of operation, the pump 121 applies suction to the catheter 14 via the tube 44 to aspirate fluid and move the aspirated fluid into the reciprocal tube 22 (i.e., the aspiration direction). Before the aspirated fluid reaches the pump 121, the controller 50 halts the pump 121 from further creating suction. In some cases, multiple individual suctions may be performed before the aspirated fluid nears the pump 121 – the number of suctions can relate to, for example, a predefined limit on volume of aspirate allowed in the reciprocal tube 22, a duration of each aspiration, and a vacuum pressure reached during each suction, among other factors. Subsequently, the controller 50 executes a second or push mode of operation and reverses the rotation of the rollers 48 to expel the aspirated fluid via the tube 44 in the clot capture direction through the reciprocal tube 22, the fitting 24, the second valve 30, and the clot capture container 28. The controller 50 can also control a rotational speed of the rollers 48, which, in conjunction with the inner diameter of the tube 44, can establish the amount of negative and positive pressure and the flow rate of the fluid in the tube 44. The tube 44 may have a consistent inner diameter throughout or may include tubing portions with different inner diameters. Accordingly, the flow rate of the fluid in the aspiration direction can be arranged to be different than the flow rate of the fluid in the clot capture direction. In an embodiment, the fluid flow rate is higher in the aspiration direction than in the clot capture direction, which would allow for a more aggressive application of suction for retrieval of the clot and a gentler application of forced flow should the system be configured to reinfuse aspirated blood into the patient.

FIGS. 5A, 5B, and 5C, illustrate non-limiting and only illustrative examples of check valves (e.g., the first valve 20 and the second valve 30) that can be used with embodiments of the present inventions. FIG. 5A exemplifies a swing check valve or tilting disc check valve that has a movable disc 52 to block the flow. The disk 52 swings on a hinge or trunnion to block reverse flow (e.g., as illustrated on the left in FIG. 5A) and to allow forward flow (e.g., as illustrated on the right in FIG. 5A). FIG. 5B exemplifies a lift-check valve in which a stop 54 is lifted by higher pressure of inlet or upstream fluid to allow flow to the outlet or downstream side. When the pressure drops, gravity or higher downstream pressure will cause the stop 54 to lower, shutting the valve to stop reverse flow (e.g., as illustrated on the left in FIG. 5B), reopening after the upstream pressure reaches or exceeds the cracking pressure (e.g., as illustrated on the right in FIG. 5B). A spring 56 can be used to regulate the degree of cracking force of the valve . FIG. 5C exemplifies a diaphragm check valve that uses a flexing rubber diaphragm 58 in which the pressure differential on the upstream side is greater than the pressure on the downstream for the check valve to open (e.g., flow allowed as illustrated on the top in FIG. 5C and reduced flow as the pressure differential decreases as illustrated on the bottom in FIG. 5C.)

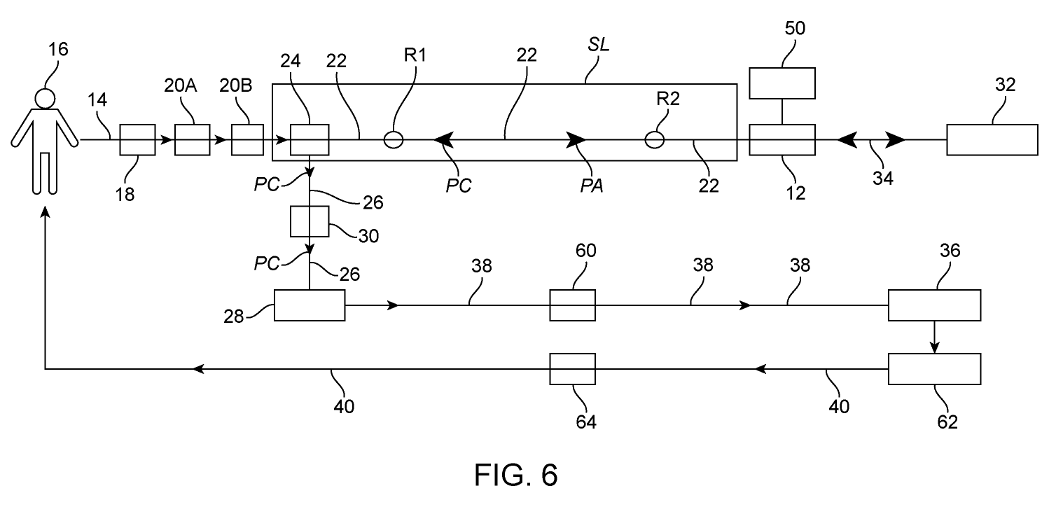

The embodiments of the system 10 of the present inventions are not limited to the aforementioned components and the illustrated configuration, and can include additional components and other configurations, an example of which is illustrated in FIG. 6. In FIG. 6, two valves 20A and 20B (e.g., check valves) are positioned in series downstream of the catheter 14. A second valve (e.g., two valves 20A and 20B instead of a single valve 20) can further safeguard against inadvertent backflow, and to ensure protection in case of failure of a valve to prevent backflow (e.g., the valve 20A is a backup to the valve 20B and vice versa.) A third valve 60 (e.g., a check valve) can be added downstream of the clot capture container 28 and upstream of the blood reservoir 36 on the blood reservoir line 38 to prevent backflow from the blood reservoir 36 into the clot capture container 28. The blood reservoir 36 can have a built-in filter included within the blood reservoir 36; additionally or alternatively a filter 62 can be connected downstream of the blood reservoir 36. A fourth valve 64 (e.g., a check valve) can be added downstream of the blood reservoir 36 or the filter 62 (if a separate filter is used) to prevent any backflow of fluid into the filter 62 and/or the blood reservoir 36.

The length of the reciprocal tube 22 can be demarcated approximately by a box labeled SL. In one embodiment, the length of the reciprocal tube 22 can end at the proximal side (closer to the pump 12) of the fitting 24, about middle of the fitting 24, or up to the branch point of the fitting 24. The box SL includes two arrows, PA and PC. The arrow PA represents pull in the aspiration direction and the arrows PC represent push in the clot capture direction. One or more sensors that can perform various sensing functions, for example R1 and/or R2, can be added to the reciprocal tube 22, and/or one or more sensors can be added on the clot capture side of the second valve. In an embodiment, the sensor R1 can be used to determine a quantity of saline in the reciprocal tube 22 to prevent over-dilution of blood with saline. In an embodiment, the sensor R2 can be positioned at a selected distance from the pump 12 to communicate a signal to the controller 50 to, for example, halt operation of the pump 12 or reverse pump function from the pull/aspiration direction mode to the push/clot capture direction mode in order to prevent aspirated fluid entering the pump 12. In an embodiment, another sensor (not shown) can be included on the clot capture side of the second valve to monitor flow rate, saline concentration, or other metric.

The controller 50 can include a CPU and a non-transitory recording medium storing a computer readable control program for halting or reversing the operation of the pump 12 once the presence of the aspirated fluid is detected at the sensor R2. The non-transitory recording medium storing a computer readable control program can cause a computer to perform a halt or a reversal of the pump function from suction to positive displacement. The program can also control the output of the pump 12 for controlling flow rates in the aspirated and clot capture directions. The sensor R2, alternatively, can communicate with a monitor to produce a visual or audio alert to the physician that the aspirated fluid is about to enter the pump 12 and that the operation of the pump 12 needs to be halted or reversed to divert the collected aspirate fluid to the clot capture direction. The saline reservoir can act as a purge tank should the aspirated fluid inadvertently by-pass the pump 12. The sensor R1 or R2, or a different sensor, can also assess the pressure in the reciprocal tube 22 which could be indicative of a clog. The controller 50 can execute pull-push cycles (e.g., short duration cycles to dislodge the clog) until pressure in the reciprocal tube 22 is normalized, indicative that the clot has been removed. In an embodiment, the pump 12 is controllable at varying speeds, which may be varied manually or automatically (e.g., by the control program).

The embodiments of the present invention provide many advantages and advancements, some of which include the following, and others which can be readily appreciated by those skilled in the art. The placement of the clot capture container 28 out of the aspiration flow path (flow in the aspiration direction) eliminates attenuation of flow in the aspiration direction between the pump 12 and the catheter 14 that would have otherwise occurred if the clot capture container 28 was in the aspiration flow path. By comparison, a coupling of the clot capture container 28 on the reciprocal tube 22 in the flow path in the aspiration direction, between the pump 12 and the catheter 14, would necessitate that the clot capture container 28 be fully charged with vacuum pressure before the catheter 14 could passably perform aspiration function. Moreover, by eliminating or minimizing damping by removing the clot capture container 28 out of flow in the aspiration direction, a large diameter reciprocal tube 22 can be used. A large diameter reciprocal tube 22 reduces the blood flow velocity through the tube, which reduces hemolysis and eliminates or minimizes the possibility of clogging in the tube. A large diameter reciprocal tube 22 additionally reduces the pressure loss along the length of the line ensuring high vacuum pressure is applied to the catheter for optimal clot aspiration.

The placement of the clot capture container 28 out of the flow path in the aspiration direction also reduces hemolysis by eliminating blood traversing through the pump 12 in either the aspiration direction or the clot capture direction. By comparison, if the blood flowed through the pump 12, the peristalsis process (i.e., the wipers or rollers 48 compressing the tube 44) would exert pressure on the blood traversing through the pump 12, which could consequently damage the cells.

Further, the configuration of the system 10 (e.g., as illustrated in FIG. 2) allows a medical team to size and/or arrange the tubing within the sterile field of boxed outline 42a (e.g., reciprocal tube 22 and clot capture line 26) to create a short path for clot to reach the clot capture chamber 28 (aspirated in the aspiration direction and then purged in the clot capture direction to the clot capture chamber 28). This advantageously permits a physician to aspirate significantly less blood from a patient – well below the industry standard maximum amount of 500 cc.

Additionally, embodiments of the present inventions (e.g., the arrangement illustrated in FIG. 2 or the arrangement illustrated in FIG. 6) can reduce a distance that blood must travel to reach the clot capture container 28 and/or the blood reservoir 36. Because shear stresses applied by inner walls of the flow path (e.g., tube 22, 26, 34, 38, 44, fitting 24, etc.) can damage blood cells, shortening the blood flow distance can allow for the capture of healthier blood, which optionally can be circulated back into the patient.

Another advantage of the pull-push extraction circuits and systems of the present inventions is a simplified sealed connection between the pump 12 and the catheter 14 with the absence of the clot capture chamber 28 from the flow path in the aspiration direction. The simplified option for connecting a single line (e.g., the tube 22) between the pump 12 and the catheter 14 reduces or eliminates unintended air ingress that may occur (e.g., from opening the clot capture chamber 28 to inspect matter captured inside) and offers optimal suction at the catheter 14.

Further, de-airing of the system 10 that may be needed can be done by purging the closed circuit with saline solution from the saline reservoir 32, rather than having to connect and disconnect added components (e.g., a syringe or additional saline line) to introduce saline for removing air.

Another advantage of the pull-push extraction circuits and systems of the present inventions is that the closed circuit can allow the saline solution to be flushed into the clot capture container 28 to clear the clot capture container 28. Thus, a physician may be able to more easily observe clots that have been captured that otherwise would have been more difficult to assess through dense and opaque biofluids. If a clot is captured, the physician can either end the procedure, aspirate more clot from the same location, or move the catheter to other locations for further clot extraction. If the physician does not see any clots in the clot capture container 28, the physician can modify the position of the catheter 14, move the catheter 14 to a different location, and/or increase the vacuum output pressure of the pump 12 in real time. Without the saline flushing of the clot capture container 28, the physician might need to open the container to determine if any clots are captured, which could compromise sterility of the system. The closed circuit further allows for a limit to an amount of saline solution to a predetermined or a measured amount of saline solution to be introduced into the closed circuit by the pump 12 to minimize dilution of the blood in the blood reservoir 36.

In an embodiment, the saline solution used and saline storage in the saline reservoir 32 are instead a different fluid, or a composition mixed with saline.

The pull-push extraction circuits and systems of the present inventions can be used for any procedure for removing obstructive matters in any bodily lumens.

It is understood that this disclosure, in many respects, is only illustrative of the numerous alternative device embodiments of the present inventions. Changes may be made in the details, particularly in matters of arrangement of various components and addition of devices without exceeding the scope of the various embodiments of the inventions. Those skilled in the art will appreciate that the example embodiments and descriptions thereof are merely illustrative of the inventions as a whole. While several principles of the inventions are made clear in the example embodiments described above, those skilled in the art will appreciate that modifications of the structure, arrangement, elements, and uses may be within the scope of the practice of the inventions, and otherwise, which are particularly adapted to specific environments and operative requirements without departing from the scope of the inventions. In addition, while certain features and elements have been described in connection with particular embodiments, those skilled in the art will appreciate that those features and elements can be combined with the other embodiments disclosed herein.

1. A circuit for removing a clot from a patient, comprising:
(a) a reciprocal tube configured to be coupled to an aspiration catheter for suctioning fluid from a patient;
(b) a pump connected to the reciprocal tube and configured to:
(i) apply suction to the aspiration catheter for aspirating the fluid in an aspiration direction along the reciprocal tube, and
(ii) pump the aspirated fluid in a clot capture direction, reverse of the aspiration direction, along the reciprocal tube to a clot capture container; and
(c) a controller for modifying operation of the pump from a first mode of operation of aspirating the fluid and displacing the aspirated fluid in the aspiration direction along the reciprocal tube, to a second mode of operation of pumping the aspirated fluid and displacing the aspirated fluid in the clot capture direction along the reciprocal tube toward the clot capture container.

2. The circuit of claim 1, additionally comprising a first valve connected to the reciprocal tube downstream of the aspiration catheter when flow is in the aspiration direction, wherein the first valve is either configured to or is adjustable to prevent backflow of the aspirated fluid from the reciprocal tube to the catheter.

3. The circuit of claim 1, additionally comprising the clot capture container connected in a flow path in the clot capture direction wherein the clot capture container is positioned in the flow path to prevent or minimize flow attenuation between the pump and the aspiration catheter, and/or minimize air ingress into an aspiration side of the circuit when the clot capture container is opened to remove or inspect clot.

4. The circuit of claim 1, additionally comprising the clot capture container connected to the reciprocal tube by a clot capture line, wherein the clot capture direction extends along the clot capture line to the clot capture container for clot to be removed from the aspirated fluid.

5. The circuit of claim 4, additionally comprising a second valve connected to the clot capture line at an upstream position of the clot capture container, wherein the second valve is either configured to or is adjustable to prevent backflow of the aspirated fluid from the clot capture line into the reciprocal tube.

6. The circuit of claim 4, additionally comprising a blood reservoir assembly connected to the clot capture container for receiving aspirated fluid, wherein the operation of the pump in the second mode of operation promotes filtered aspirated fluid to be reinfused into the patient from the blood reservoir assembly.

7. The circuit of claim 6, additionally comprising a third valve positioned between the clot capture container and the blood reservoir assembly to prevent backflow of the aspirated fluid into the clot capture container.

8. The circuit of claim 1, wherein the pump is a peristaltic pump.

9. The circuit of claim 1, wherein the pump is connected to a saline reservoir by a saline line with an inner diameter greater than 1/8 inch, and wherein the circuit is configured to allow the pump to purge the reciprocal tube with saline solution from the saline reservoir during the pumping of the aspirated fluid in the clot capture direction.

10. The circuit of claim 1, wherein the circuit is a closed circuit to allow de-airing of the reciprocal tube with saline solution by pumping saline solution from a saline reservoir connected to the pump through the reciprocal tube.

11. The circuit of claim 1, further comprising the aspiration catheter.

12. The circuit of claim 1, additionally comprising a blood reservoir assembly connected to the clot capture container for receiving aspirated fluid and a filter positioned with the blood reservoir or downstream of the blood reservoir to remove clot or particulate from the aspirated blood.

13. A system for removing a clot from a patient, comprising:
(a) a network of tubes for allowing blood and clot aspirated from a patient to move in (i) an aspiration direction, and (ii) a clot capture direction for depositing the clot in a clot capture container; and
(b) a pump configured to independently control the flow rate of the blood in the aspiration direction and the clot capture direction, such that the flow rate of the blood in the aspiration direction is different than the flow rate of the blood in the clot capture direction.

14. The system of claim 13, wherein the clot capture direction is in an opposite direction of the aspiration direction along at least a segment of a same tube.

15. The system of claim 13, wherein the aspiration direction is from a catheter toward the pump along a segment of a tube connecting the catheter to the pump, and the clot capture direction is in an opposite direction of the aspiration direction along at least a part of a same segment of the tube.

16. The system of claim 13, wherein the flow rate of the blood in the clot capture direction is set to minimize hemolysis by reducing a velocity of blood delivered to a blood reservoir.

17. The system of claim 13, wherein the pump is a peristaltic pump, the operation of which can be continuously switched between pulling and pushing fluid in the aspiration direction and clot capture direction, respectively.

18. A method of removing a clot from a patient, comprising:
positioning a catheter at a target site of the patient;
aspirating fluid from the catheter into a reciprocal tube; and
reversing the flow of aspirated fluid flowing along the reciprocal tube to reroute the aspirated fluid toward a clot capture container for capturing a clot.

19. The method of claim 18, additionally comprising continuously repeated aspirating fluid and reversing the flow of the aspirated fluid to remove more clot from the patient.

20. The method of claim 18, wherein the flow of aspirated fluid is reversed prior to the aspirated fluid reaching a pump used to suction the aspirated fluid from a catheter.

21. The method of claim 20, wherein the pump is further used to pump the aspirated fluid into the clot capture container in a clot capture direction.

22. The method of claim 18, additionally comprising:
(a) pumping aspirated fluid through the clot capture container and into a blood reservoir assembly in which the aspirated fluid can be filtered for reinfusion into the patient; and/or
(b) suctioning saline solution from a saline reservoir when the flow of aspirated fluid is reversed to purge the aspirated fluid from the reciprocal tube into the clot capture container.

23. A clot extraction system, comprising:
(a) a first tube having a distal end coupled to a catheter;
(b) a clot capture container coupled by a second tube to the first tube; and
(c) a pump connected to a proximal end of the first tube and configured to apply negative pressure to the first tube to aspirate a clot from the patient through the catheter and to apply positive pressure to the first tube to transport the clot to the clot capture container through the second tube.

24. The clot extraction system of claim 23, further comprising the catheter.

25. The clot extraction system of claim 23, additionally comprising a first valve connected to the first tube downstream of the catheter to prevent backflow of fluid from the first tube into the catheter.

26. The clot extraction system of claim 23, additionally comprising a second valve connected to the second tube to prevent backflow of fluid from the second tube into the first tube.

27. The clot extraction system of claim 23, additionally comprising a controller for controlling the operation of the pump from a first mode of operation of applying negative pressure to the first tube to aspirate the clot from the patient through the catheter to a second mode of operation of applying no pressure or positive pressure to the first tube.

28. The clot extraction system of claim 23, additionally comprising a saline reservoir connected to the pump for supplying a saline solution to the first tube.

29. The clot extraction system of claim 23, additionally comprising a blood reservoir connected to the clot capture container for receiving fluid from the clot capture container.

30. The clot extraction system of claim 29, additionally comprising a filter to filter blood in the blood reservoir and a third tube for reinfusing the filtered blood into the patient.
